(19) United States
(12) Patent Application Publication
(10) Pub. No.: US 2022/0057233 A1
(43) Pub. Date: Feb. 24, 2022

(54) MOTION STATE MONITORING SYSTEM, TRAINING SUPPORT SYSTEM, METHOD FOR CONTROLLING MOTION STATE MONITORING SYSTEM, AND CONTROL PROGRAM

(71) Applicant: Toyota Jidosha Kabushiki Kaisha, Toyota-shi Aichi-ken (JP)

(72) Inventors: Makoto Kobayashi, Nisshin-shi Aichi-ken (JP); Toru Miyagawa, Seto-shi Aichi-ken (JP); Issei Nakashima, Toyota-shi Aichi-ken (JP); Keisuke Suga, Nagoya-shi Aichi-ken (JP); Masayuki Imaida, Ichinomiya-shi Aichi-ken (JP); Manabu Yamamoto, Toyota-shi Aichi-ken (JP); Toshiaki Okumura, Toyota-shi Aichi-ken (JP); Yohei Otaka, Kariya-shi Aichi-ken (JP); Masaki Katoh, Nagoya-shi Aichi-ken (JP); Asuka Hirano, Nagoya-shi Aichi-ken (JP); Taiki Yoshida, Nagoya-shi Aichi-ken (JP)

(21) Appl. No.: 17/403,305

(22) Filed: Aug. 16, 2021

(30) Foreign Application Priority Data
Aug. 18, 2020 (JP) 2020-138238

(51) Int. Cl. G01C 25/00; G06F 3/0484

(52) U.S. Cl. CPC G01C 25/00; G01C 19/00; G06F 3/04842

(57) ABSTRACT

A motion state monitoring system including: a selection unit that selects one or a plurality of sensors from among a plurality of sensors associated with a plurality of respective body parts of a body of a subject based on one or a plurality of specified motions to be monitored; a calibration result determination unit that determines whether or not a calibration of each of at least the one or plurality of sensors selected by the selection unit has been completed; a calculation processing unit that generates a result of a calculation indicating a motion state of the subject based on a result of detection performed by each of the one or plurality of sensors selected by the selection unit when the calibration result determination unit determines that the calibration has been completed; and an output unit that outputs the result of the calculation performed by the calculation processing unit.

### CROSS REFERENCE TO RELATED APPLICATIONS

This application is based upon and claims the benefit of priority from Japanese patent application No. 2020-138238, filed on Aug. 18, 2020, the disclosure of which is incorporated herein in its entirety by reference.

### BACKGROUND

The present disclosure relates to a motion state monitoring system, a training support system, a method for controlling the motion state monitoring system, and a control program.

The motion detection apparatus disclosed in Japanese Unexamined Patent Application Publication No. 2020-81413 includes: a posture detection unit that detects, by using measurement data of a set of sensors (an acceleration sensor and an angular velocity sensor) attached to a body part of a body of a user (a subject), a posture of the body part; a time acquisition unit that acquires a time elapsed from when measurement of a motion is started; and a motion state detection unit that detects a motion state of the user by using the posture detected by the posture detection unit and the elapsed time acquired by the time acquisition unit.

### SUMMARY

However, the motion detection apparatus disclosed in Japanese Unexamined Patent Application Publication No. 2020-81413 has a problem that since the motion state of the user is detected using measurement data of only a set of sensors attached to the body part of the body of the user (the subject), a more complicated motion state of the user cannot be effectively monitored.

The present disclosure has been made in view of the aforementioned circumstances and an object thereof is to provide a motion state monitoring system, a training support system, a method for controlling the motion state monitoring system, and a control program that are capable of effectively monitoring a complicated motion state of a subject by monitoring a motion state of the subject using a result of detection performed by one or a plurality of sensors that are selected from among a plurality of sensors based on a motion to be monitored.

A first exemplary aspect is a motion state monitoring system including: a selection unit configured to select one or a plurality of sensors from among a plurality of sensors associated with a plurality of respective body parts of a body of a subject based on one or a plurality of specified motions to be monitored; a calibration result determination unit configured to determine whether or not a calibration of each of at least the one or plurality of sensors selected by the selection unit has been completed; a calculation processing unit configured to generate a result of a calculation indicating a motion state of the subject based on a result of detection performed by each of the one or plurality of sensors selected by the selection unit when the calibration result determination unit determines that the calibration has been completed; and an output unit configured to output the result of the calculation performed by the calculation processing unit. By using a result of detection performed by one or a plurality of sensors selected from among a plurality of sensors associated with a plurality of respective body parts based on a motion to be monitored, this motion state monitoring system can output a more accurate result of a calculation indicating a motion state of the subject than when a result of detection performed by a set of sensors attached to one body part is used. As a result, a user can effectively monitor a complicated motion state of the subject. Further, this motion state monitoring system can output a more accurate result of a calculation by using a result of detection performed by the sensor for which a calibration has been completed. As a result, a user can more accurately monitor the motion state of the subject.

The calibration result determination unit is configured to determine that the calibration has been completed when an output value of each of at least the one or plurality of sensors selected by the selection unit falls within a predetermined range after a predetermined period of time has elapsed from when the calibration of each of at least the one or plurality of sensors is started.

A time at which the calibration is started may be a time at which an instruction that the calibration is to be started has been given in a state in which each of at least the one or plurality of sensors selected by the selection unit has been brought to a standstill. Further, the time at which the calibration is started may be a time at which power of each of at least the one or plurality of sensors selected by the selection unit is turned on in a state in which each of at least the one or plurality of sensors has been brought to a standstill.

It is desirable that the output unit be further configured to output, when the calibration of one of at least the one or plurality of sensors selected by the selection unit is not completed, information prompting a user to bring the sensor for which the calibration is not completed to a standstill. By this configuration, a user can determine for which the sensor the calibration is not completed and bring this sensor to a standstill.

The calibration result determination unit is further configured to determine whether or not all the calibrations of the plurality of sensors associated with the plurality of respective body parts of the body of the subject have been completed. By this configuration, it is possible to complete the calibration before pairing is performed.

Another exemplary aspect is a training support system including: a plurality of measuring instruments each including one of the plurality of sensors associated with a plurality of respective body parts of a body of a subject; and the motion state monitoring system according to any one of the above-described aspects. By using a result of detection performed by one or a plurality of sensors selected from among a plurality of sensors associated with a plurality of respective body parts based on a motion to be monitored, this training support system can output a more accurate result of a calculation indicating a motion state of the subject than when a result of detection performed by a set of sensors attached to one body part is used. As a result, a user can effectively monitor a complicated motion state of the subject. Further, this training support system can output a more accurate result of a calculation by using a result of detection performed by the sensor for which a calibration has been completed. As a result, a user can more accurately monitor the motion state of the subject.

Another exemplary aspect is a method for controlling a motion state monitoring system, the method including: selecting one or a plurality of sensors from among a plurality of sensors associated with a plurality of respective body parts of a body of a subject based on one or a plurality of specified motions to be monitored; determining whether or not a calibration of each of at least the one or plurality of selected sensors has been completed; generating a result of a calculation indicating a motion state of the subject based on a result of detection performed by each of the one or plurality of selected sensors when it is determined that the calibration has been completed; and outputting the result of the calculation. In this method for controlling a motion state monitoring system, by using a result of detection performed by one or a plurality of sensors selected from among a plurality of sensors associated with a plurality of respective body parts based on a motion to be monitored, it is possible to output a more accurate result of a calculation indicating a motion state of the subject than when a result of detection performed by a set of sensors attached to one body part is used. As a result, a user can effectively monitor a complicated motion state of the subject. Further, in this method for controlling a motion state monitoring system, it is possible to output a more accurate result of a calculation by using a result of detection performed by the sensor for which a calibration has been completed. As a result, a user can more accurately monitor the motion state of the subject.

Another exemplary aspect is a control program for causing a computer to: select one or a plurality of sensors from among a plurality of sensors associated with a plurality of respective body parts of a body of a subject based on one or a plurality of specified motions to be monitored; determine whether or not a calibration of each of at least the one or plurality of selected sensors has been completed; generate a result of a calculation indicating a motion state of the subject based on a result of detection performed by each of the one or plurality of selected sensors when it is determined that the calibration has been completed; and output the result of the calculation. By using a result of detection performed by one or a plurality of sensors selected from among a plurality of sensors associated with a plurality of respective body parts based on a motion to be monitored, this control program can output a more accurate result of a calculation indicating a motion state of the subject than when a result of detection performed by a set of sensors attached to one body part is used. As a result, a user can effectively monitor a complicated motion state of the subject. Further, this control program can output a more accurate result of a calculation by using a result of detection performed by the sensor for which a calibration has been completed. As a result, a user can more accurately monitor the motion state of the subject.

According to the present disclosure, it is possible to provide a motion state monitoring system, a training support system, a method for controlling the motion state monitoring system, and a control program that are capable of effectively monitoring a complicated motion state of a subject by monitoring a motion state of the subject using a result of detection performed by one or a plurality of sensors that are selected from among a plurality of sensors based on a motion to be monitored.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration example of a training support system according to a first embodiment;

FIG. 2 is a diagram showing an example of body parts to which measuring instruments are to be attached;

FIG. 3 is a diagram showing a configuration example of the measuring instrument provided in the training support system shown in FIG. 1;

FIG. 4 is a diagram showing an example of how to attach the measuring instrument shown in FIG. 3;

FIG. 5 is a diagram for explaining a calibration;

FIG. 6 is a flowchart showing an operation of the training support system shown in FIG. 1;

FIG. 7 is a diagram showing an example of a screen (a selection screen of a motion to be monitored) displayed on a monitor;

FIG. 8 is a diagram showing an example of the screen (the selection screen of the motion to be monitored) displayed on the monitor;

FIG. 9 is a diagram showing an example of the screen (the selection screen of the motion to be monitored) displayed on the monitor;

FIG. 10 is a diagram showing an example of the screen (the selection screen of the motion to be monitored) displayed on the monitor;

FIG. 11 is a diagram showing an example of a screen (a screen during the calibration) displayed on the monitor;

FIG. 12 is a diagram showing an example of a screen (a screen after the calibration has been completed) displayed on the monitor;

FIG. 13 is a diagram showing an example of a screen (a screen before measurement) displayed on the monitor;

FIG. 14 is a diagram showing an example of a screen (a screen during the measurement) displayed on the monitor;

FIG. 15 is a block diagram showing a modified example of the training support system shown in FIG. 1; and

FIG. 16 is a diagram showing a configuration example of a measuring instrument provided in a training support system shown in FIG. 15.

### DESCRIPTION OF EMBODIMENTS

Hereinafter, although the present disclosure will be described with reference to an embodiment of the present disclosure, the present disclosure according to claims is not limited to the following embodiment. In order to clarify the explanation, the following descriptions and the drawings are partially omitted and simplified as appropriate. Further, the same symbols are assigned to the same elements throughout the drawings, and redundant descriptions are omitted as necessary.

### First Embodiment

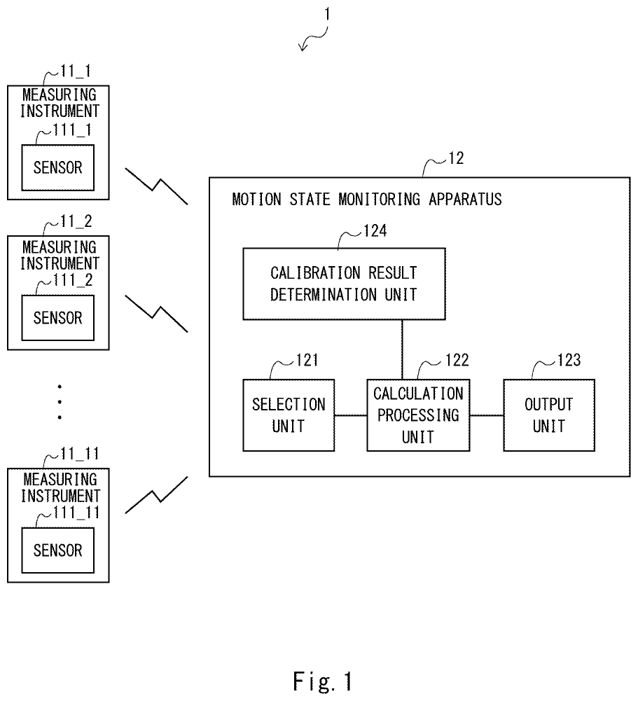

FIG. 1 is a block diagram showing a configuration example of a training support system 1 according to a first embodiment. The training support system 1 is a system for monitoring a motion of a subject and providing support for making the motion of the subject close to a desired motion based on a result of the monitoring. The details thereof will be described below.

As shown in FIG. 1, the training support system 1 includes a plurality of measuring instruments 11 and a motion state monitoring apparatus 12. In this embodiment, an example in which 11 of the measuring instruments 11 are provided will be described. In the following description, the 11 measuring instruments 11 are also referred to as measuring instruments 11_1 to 11_11, respectively, in order to distinguish them from each other.

The measuring instruments 11_1 to 11_11 are respectively attached to body parts 20_1 to 20_11 from which motions are to be detected among various body parts of the body of a subject P, and detect the motions of the respective body parts 20_1 to 20_11 by using motion sensors (hereinafter simply referred to as sensors) 111_1 to 111_11 such as gyro sensors. Note that the measuring instruments 11_1 to 11_11 are associated with the respective body parts 20_1 to 20_11 by pairing processing performed with the motion state monitoring apparatus 12.

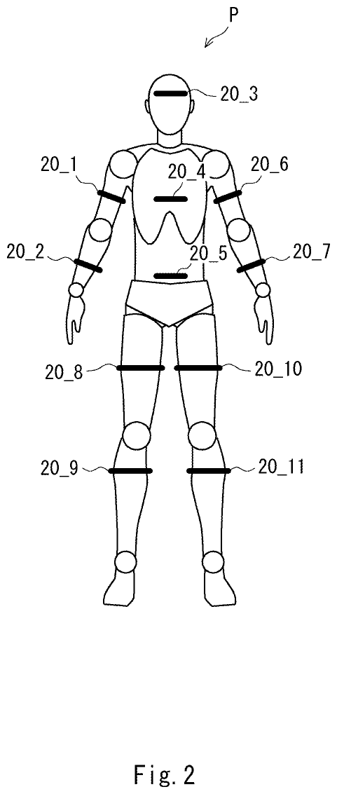

FIG. 2 is a diagram showing an example of the body parts to which the measuring instruments 11_1 to 11_11 are to be attached. In the example shown in FIG. 2, the body parts 20_1 to 20_11 to which the respective measuring instruments 11_1 to 11_11 are to be attached are a right upper arm, a right forearm, a head, a chest (a trunk), a waist (a pelvis), a left upper arm, a left forearm, a right thigh, a right lower leg, a left thigh, and a left lower leg, respectively.

### (Configuration Examples of Measuring Instruments 11_1 to 11_11)

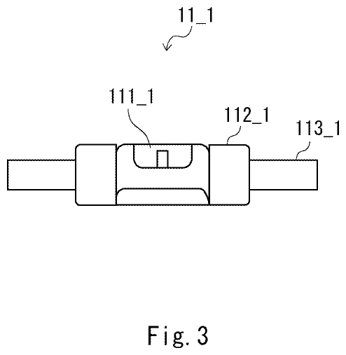

FIG. 3 is a diagram showing a configuration example of the measuring instrument 11_1. Note that the configuration of each of the measuring instruments 11_2 to 11_11 is similar to that of the measuring instrument 11_1, and thus the descriptions thereof will be omitted.

As shown in FIG. 3, the measuring instrument 11_1 includes a sensor 111_1, an attachment pad 112_1, and a belt 113_1. The belt 113_1 is configured so that it can be wound around the body part of the subject P from which a motion is to be detected. The sensor 111_1 is integrated with, for example, the attachment pad 112_1. Further, the attachment pad 112_1 is configured so that it can be attached to or detached from the belt 113_1.

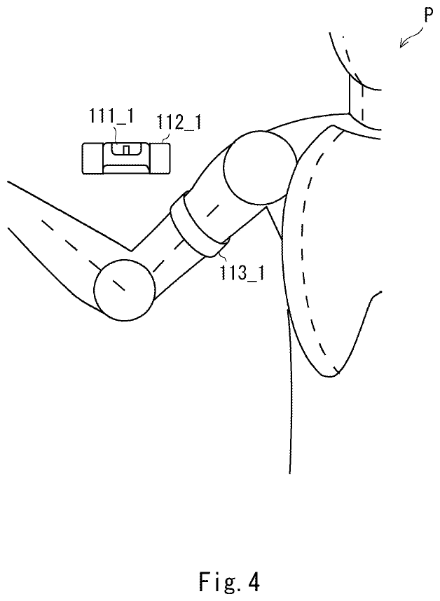

FIG. 4 is a diagram showing an example of how to attach the measuring instrument 11_1. In the example shown in FIG. 4, the belt 113_1 is wound around the right upper arm which is one of the body parts of the subject P from which motions are to be detected. The sensor 111_1 is attached to the belt 113_1 with the attachment pad 112_1 interposed therebetween after pairing, a calibration, and the like have been completed.

Referring back to FIG. 1, the description will be continued.

The motion state monitoring apparatus 12 is an apparatus that outputs a result of a calculation indicating a motion state of the subject P based on results (sensing values) of detection performed by the sensors 111_1 to 111_11. The motion state monitoring apparatus 12 is, for example, one of a Personal Computer (PC), a mobile phone terminal, a smartphone, and a tablet terminal, and is configured so that it can communicate with the sensors 111_1 to 111_11 via a network (not shown). The motion state monitoring apparatus 12 can also be referred to as a motion state monitoring system.

Specifically, the motion state monitoring apparatus 12 includes at least a selection unit 121, a calculation processing unit 122, an output unit 123, and a calibration result determination unit 124.

The selection unit 121 selects, from among the sensors 111_1 to 111_11 associated with the respective body parts 20_1 to 20_11 of the body of the subject P, one or a plurality of sensors used to measure a motion (a motion such as bending and stretching the right elbow and internally and externally rotating the left shoulder) to be monitored which is specified by a user such as an assistant.

The calibration result determination unit 124 determines whether or not calibrations of at least the one or plurality of sensors selected by the selection unit 121 have been completed.

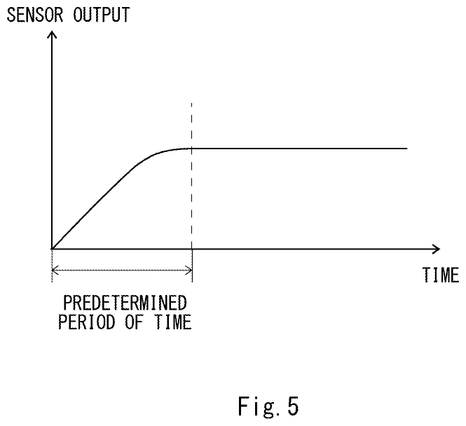

A calibration is, for example, processing for measuring an output value (an error component) of a sensor in a standstill state, the sensor being used to measure a motion to be monitored, and subtracting the error component from a measured value. It should be noted that the output value of the sensor is stabilized within a predetermined range after about 20 seconds has elapsed from when the sensor is brought to a standstill (see FIG. 5). Therefore, in the calibration, it is desirable that the output value of the sensor after a predetermined period of time (e.g., 20 seconds) has elapsed from when the sensor is brought to a standstill be used as an error component. In this embodiment, a description will be given of an example in which the output value of the sensor after a predetermined period of time has elapsed from when a user has given an instruction to start the calibration after the sensor has been brought to a standstill is used as an error component. However, a time at which the calibration is started is not limited to the time at which the user has given an instruction to start the calibration after the sensor has been brought to a standstill, and may be, for example, the time at which the power of the sensor is turned on after the sensor has been brought to a standstill. Further, “during the calibration” means a processing period of time until an error component is determined, and “completion of the calibration” means that the output value (the error component) of the sensor in a standstill state has been determined.

The calculation processing unit 122 performs calculation processing based on a result of detection performed by each of the one or plurality of sensors selected by the selection unit 121, and generates a result of the calculation indicating a motion state of the motion to be monitored. It should be noted that the calculation processing unit 122 performs the aforementioned calculation processing when the calibration result determination unit 124 determines that the calibration has been completed. By doing so, the calculation processing unit 122 can prevent the result of detection performed by the sensor that has not been calibrated from being used erroneously.

The output unit 123 outputs a result of the calculation performed by the calculation processing unit 122. The output unit 123 is, for example, a display apparatus, and displays a result of a calculation performed by the calculation processing unit 122 on a monitor, for example, by graphing the result. In this embodiment, an example in which the output unit 123 is a display apparatus will be described. However, the output unit 123 is not limited to being a display apparatus, and may instead be a speaker for outputting by voice a result of a calculation performed by the calculation processing unit 122, or a transmission apparatus that transmits a result of a calculation performed by the calculation processing unit 122 to an external display apparatus or the like.

Further, the output unit 123 may be configured to output a result of determination performed by the calibration result determination unit 124. For example, the output unit 123 may output information indicating that the calibration has been completed, and when the calibration is not completed even after a predetermined period of time has elapsed, it may output information prompting a user to bring the sensor for which the calibration is not completed to a standstill.

### (Operation of Training Support System 1)

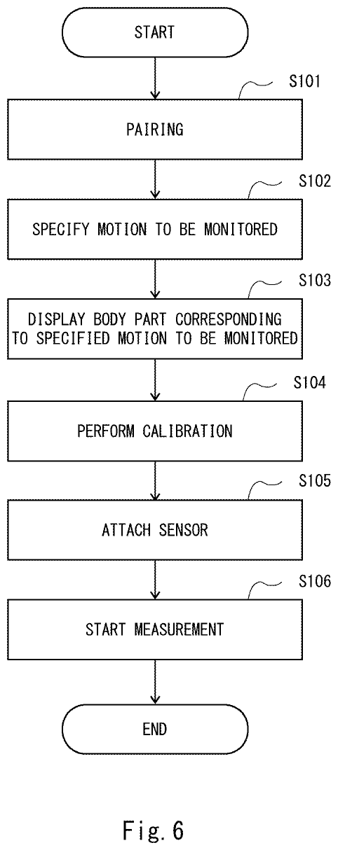

FIG. 6 is a flowchart showing an operation of the training support system 1.

In the training support system 1, pairing processing is first performed between the measuring instruments 11_1 to 11_11 and the motion operation state monitoring apparatus 12, whereby the measuring instruments 11_1 to 11_11 and the body parts 20_1 to 20_11 are respectively associated with each other (Step S101). Note that the pairing processing can also be performed in advance by previously registering the above respective measuring instruments and body parts.

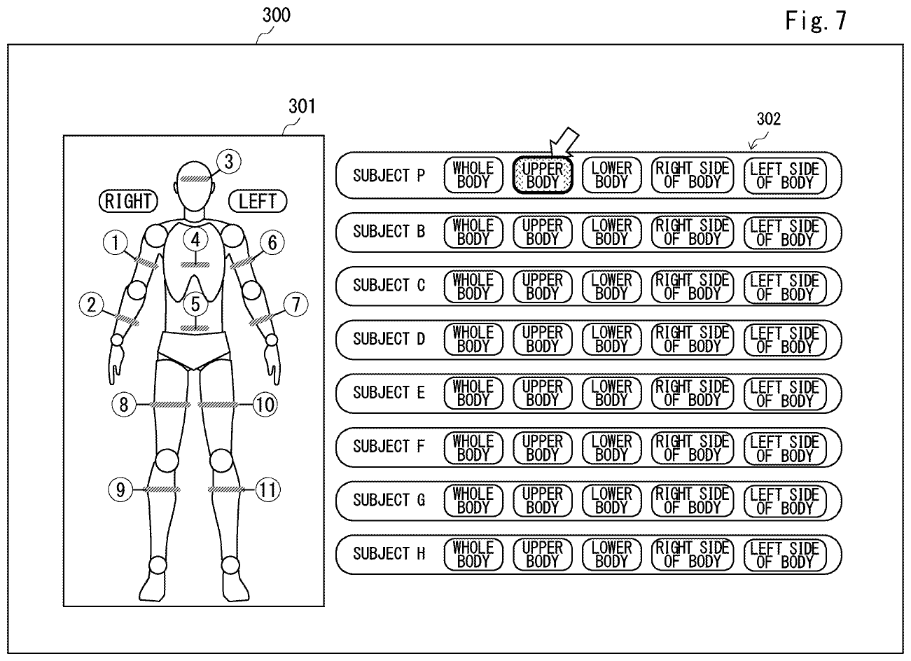

After that, a user specifies a motion to be monitored of the subject P (Step S102). This allows the output unit 123, which is a display apparatus, to display the body part to which the sensor used to measure the specified motion to be monitored is to be attached (Step S103). A method for a user to specify a motion to be monitored will be described below with reference to FIGS. 7 to 10. FIGS. 7 to 10 are diagrams each showing an example of a screen displayed on a monitor 300 of the output unit 123 which is the display apparatus.

As shown in FIG. 7, a list 302 of a plurality of subjects and a human body schematic diagram 301 showing a body part to which the sensor is to be attached are first displayed on the monitor 300. Note that “1” to “11” shown in the human body schematic diagram 301 correspond to the body parts 20_1 to 20_11, respectively. In the example shown in FIG. 7, a user has selected the subject P as a subject to be monitored. Further, the user has selected the “upper body” of the subject P as the body part for which motions are to be monitored.

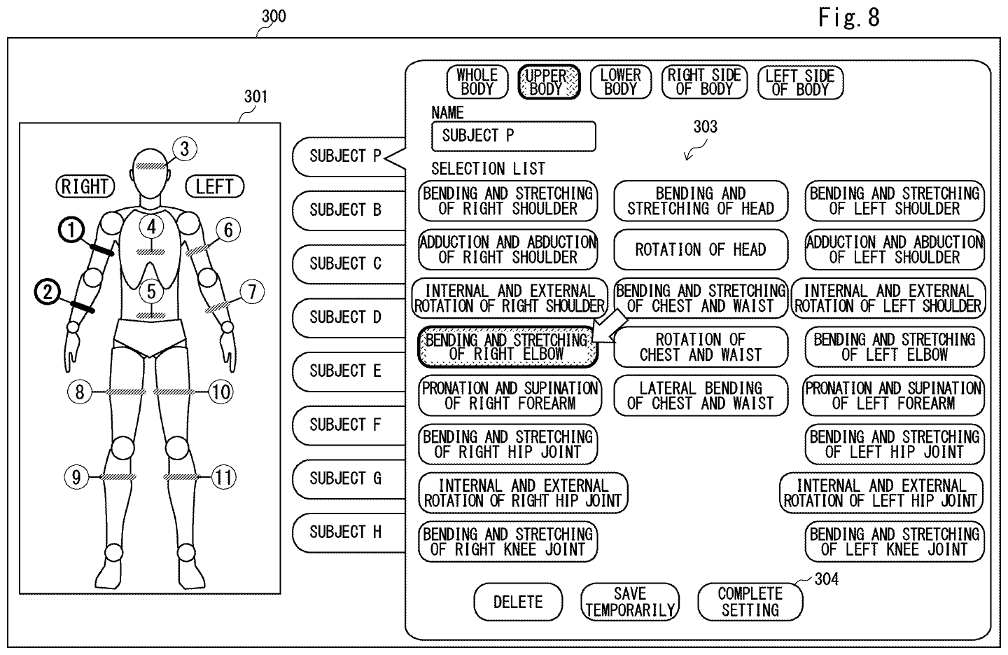

After that, as shown in FIG. 8, the monitor 300 displays a selection list 303 in which more detailed motions to be monitored are listed from among motions regarding the “upper body” of the subject P selected as the body part for which the motions are to be monitored.

This selection list 303 includes, for example, motions such as bending and stretching of the right shoulder, adduction and abduction of the right shoulder, internal and external rotation of the right shoulder, bending and stretching of the right elbow, pronation and supination of the right forearm, bending and stretching of the head, rotation of the head, bending and stretching of the chest and the waist, rotation of the chest and the waist, lateral bending of the chest and the waist, bending and stretching of the left shoulder, adduction and abduction of the left shoulder, internal and external rotation of the left shoulder, bending and stretching of the left elbow, and pronation and supination of the left forearm. The user selects more detailed motions to be monitored from this selection list 303. By doing so, among the body parts “1” to “11” (the body parts 20_1 to 20_11) to which the sensors are to be attached shown in the human body schematic diagram 301, the body part to which the sensor used to measure the motions to be monitored specified by the user is to be attached is highlighted.

In the example shown in FIG. 8, the user has selected the “bending and stretching of the right elbow” from the selection list 303. Here, it is possible to measure the bending and stretching motion of the right elbow based on a result of the detection performed by each of the sensor (111_1) attached to the right upper arm (the body part 20_1) and the sensor (111_2) attached to the right forearm (the body part 20_2). Therefore, in the example shown in FIG. 8, the body parts “1” and “2” (the body parts 20_1 and 20_2), which are body parts to which the sensors are to be attached, are highlighted, the sensors being used to measure the “bending and stretching of the right elbow” which is the motion to be monitored. After the user selects the motions from the selection list 303, he/she presses a setting completion button 304.

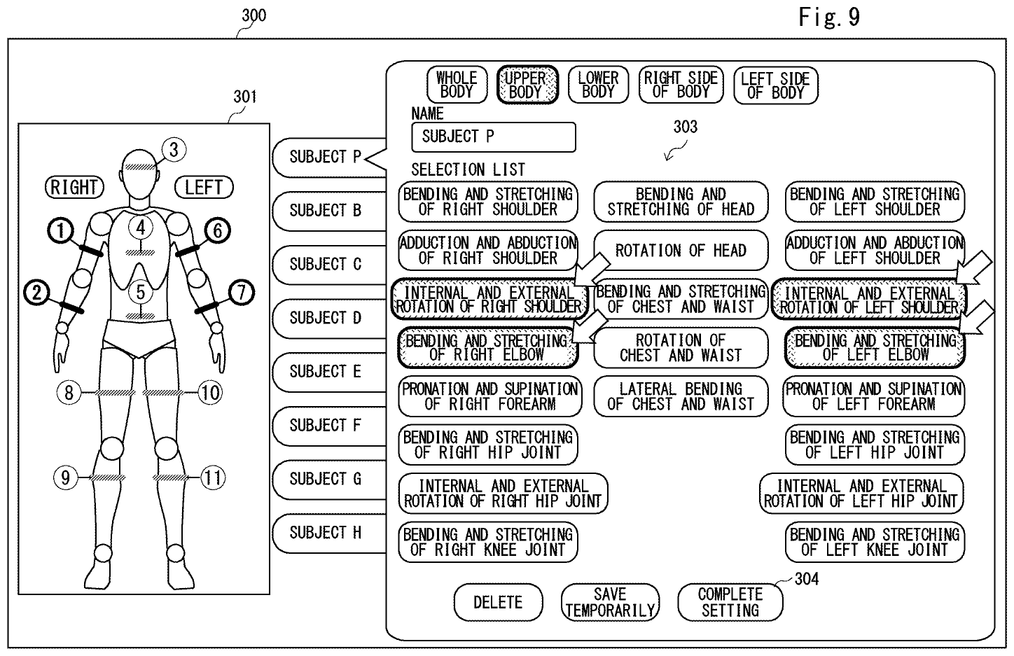

Note that, in the example of FIG. 8, although only the “bending and stretching of the right elbow” has been selected as the motion to be monitored, this is merely an example, and a plurality of motions to be monitored may instead be selected as shown in the example of FIG. 9.

In the example shown in FIG. 9, a user has selected the “bending and stretching of the right elbow”, the “internal and external rotation of the right shoulder”, the “bending and stretching of the left elbow”, and the “internal and external rotation of the left shoulder” from the selection list 303.

Here, it is possible to measure the bending and stretching motion of the right elbow based on a result of the detection performed by each of the sensor (111_1) attached to the right upper arm (the body part 20_1) and the sensor (111_2) attached to the right forearm (the body part 20_2). Similarly, it is possible to measure the internal and external rotation motion of the right shoulder based on the result of the detection performed by each of the sensor (111_1) attached to the right upper arm (the body part 20_1) and the sensor (111_2) attached to the right forearm (the body part 20_2).

Further, it is possible to measure the bending and stretching motion of the left elbow based on a result of the detection performed by each of the sensor (111_6) attached to the left upper arm (the body part 20_6) and the sensor (111_7) attached to the left forearm (the body part 20_7). Similarly, it is possible to measure the internal and external rotation motion of the left shoulder based on the result of the detection performed by each of the sensor (111_6) attached to the left upper arm (the body part 20_6) and the sensor (111_7) attached to the left forearm (the body part 20_7).

Therefore, in the example shown in FIG. 9, the body parts “1”, “2”, “6”, and “7” (the body parts 20_1, 20_2, 20_6, and 20_7), which are body parts to which the sensors are to be attached, are highlighted, the sensors being used to measure the “bending and stretching of the right elbow”, the “internal and external rotation of the right shoulder”, the “bending and stretching of the left elbow”, and the “internal and external rotation of the left shoulder” which are the motions to be monitored. A description will be given below of an example in which the “bending and stretching of the right elbow”, the “internal and external rotation of the right shoulder”, the “bending and stretching of the left elbow”, and the “internal and external rotation of the left shoulder” are selected as the motions to be monitored.

Note that when there is a sensor of which the power is off among the sensors used to measure the motions to be monitored, the sensor (more specifically, the body part to which the sensor of which the power is off is to be attached) of which the power is off may be highlighted.

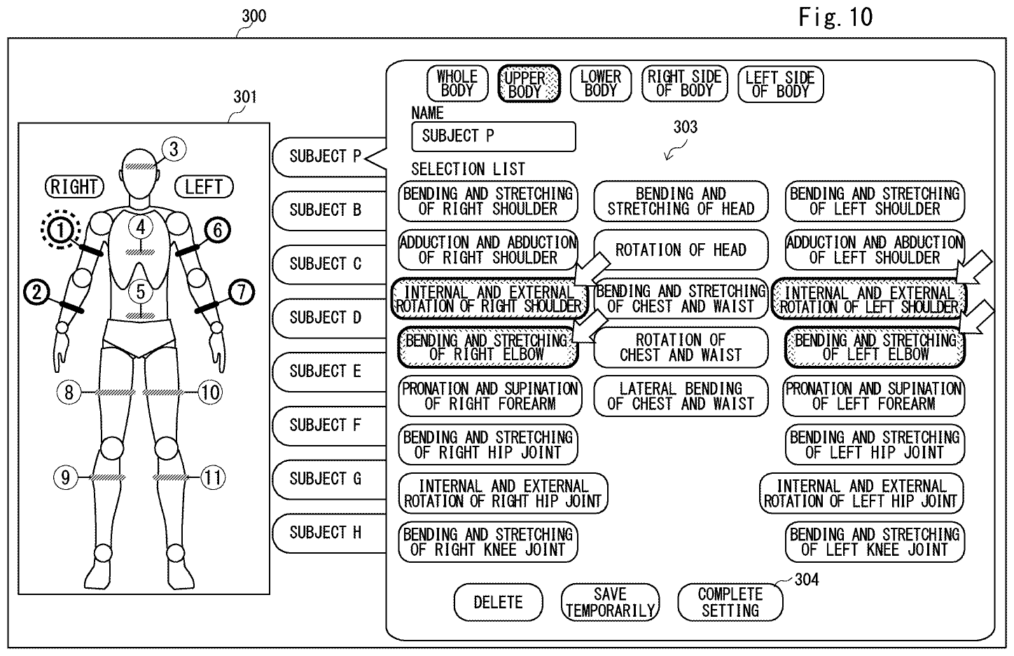

Specifically, in the example shown in FIG. 10, since the power of the sensor 111_1 is off, the body part “1” (the body part 20_1) to which the sensor 111_1 is to be attached is highlighted. Thus, a user can turn on the power of the sensor 111_1 of which the power is off or replace it with another sensor before the start of measurement of the motion to be monitored.

After the motion to be monitored is specified (Step S102) and the body part to which the sensor used to measure the motion to be monitored is to be attached is displayed (Step S103), a calibration of the sensor used to measure the motion to be monitored is subsequently performed (Step S104).

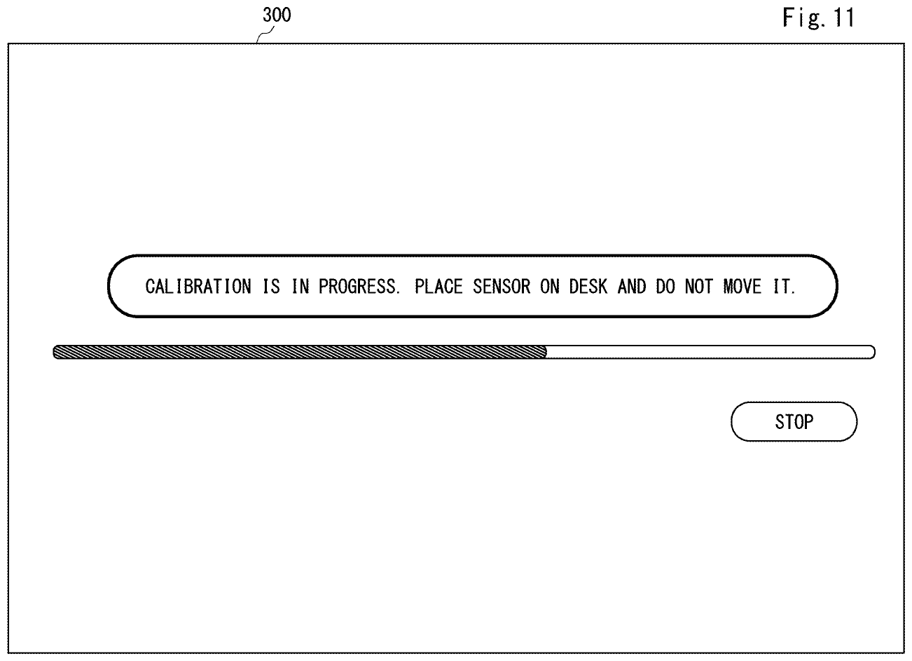

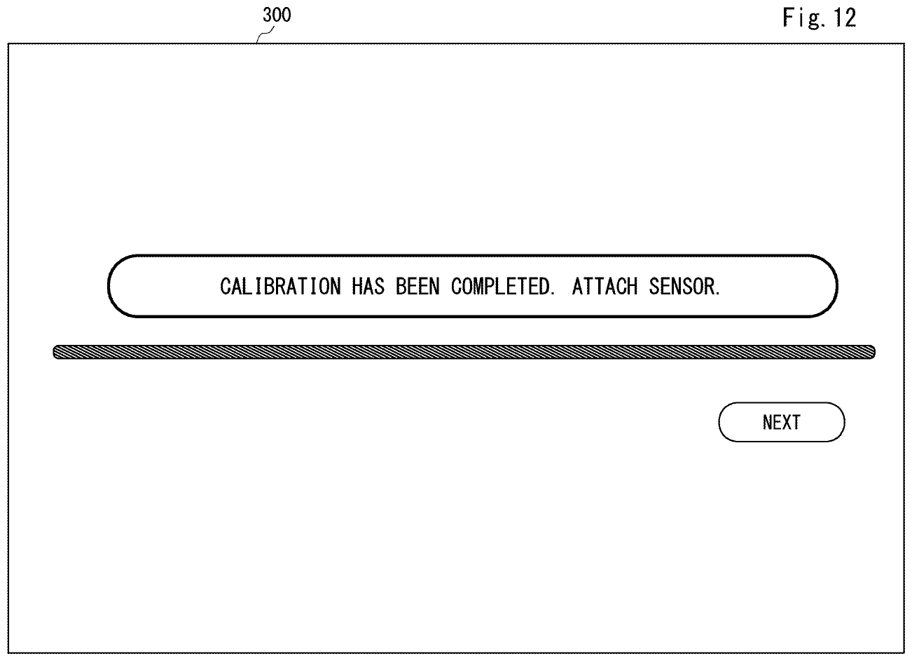

During the calibration, the monitor 300 displays, for example, the information that “Calibration is in progress. Place the sensor on the desk and do not move it” as shown in FIG. 11. Upon completion of the calibration, the monitor 300 displays, for example, the information that “Calibration has been completed. Attach the sensor” as shown in FIG. 12. Note that the information indicating that the calibration is in progress or that the calibration has been completed is not limited to being given by displaying it on the monitor 300, and may instead be given by other notification methods such as by voice. Note that when the calibration is not completed even after a predetermined period of time has elapsed, the monitor 300 displays, for example, information prompting a user to bring the sensor for which the calibration is not completed to a standstill, and therefore the user cannot proceed to the next operation until the calibration is completed.

In this example, a calibration is performed on the sensors 111_1, 111_2, 111_6, and 111_7 used to measure the motions to be monitored. However, the calibration is not limited to being performed on the sensors used to measure the motions to be monitored, and may instead be performed on all the sensors 111_1 to 111_11, for example, before the pairing processing. Note that it is only required that the calibration be completed before the start of measurement of the motion to be monitored.

After the calibration has been completed, the sensor is attached to the subject P (Step S105). In this example, the sensors 111_1, 111_2, 111_6, and 111_7 are attached to the body parts 20_1, 20_2, 20_6, and 20_7 of the subject P, respectively.

After that, the motion to be monitored is measured based on a result of detection performed by each of the sensors 111_1, 111_2, 111_6, and 111_7 (Step S106).

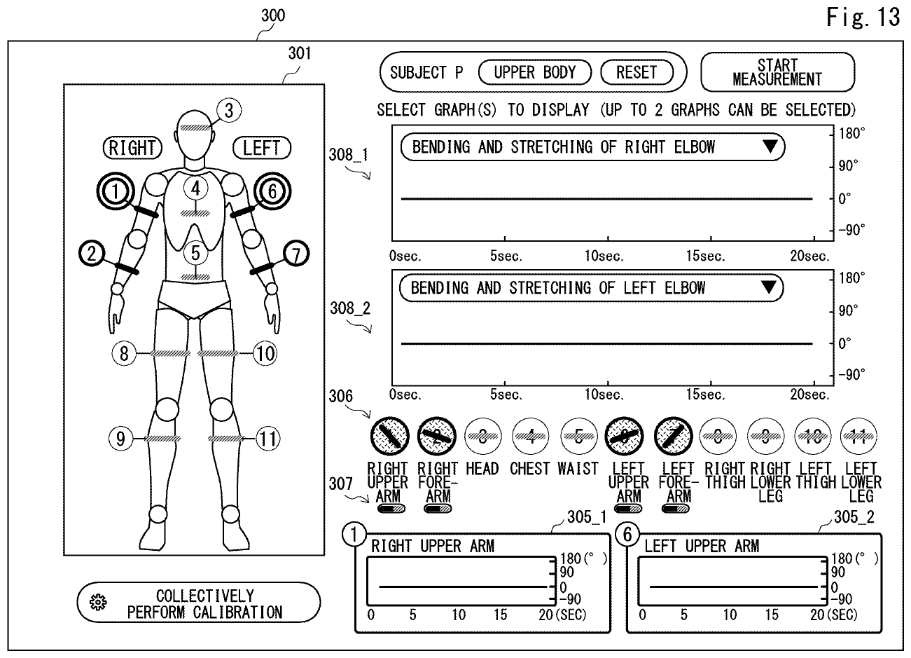

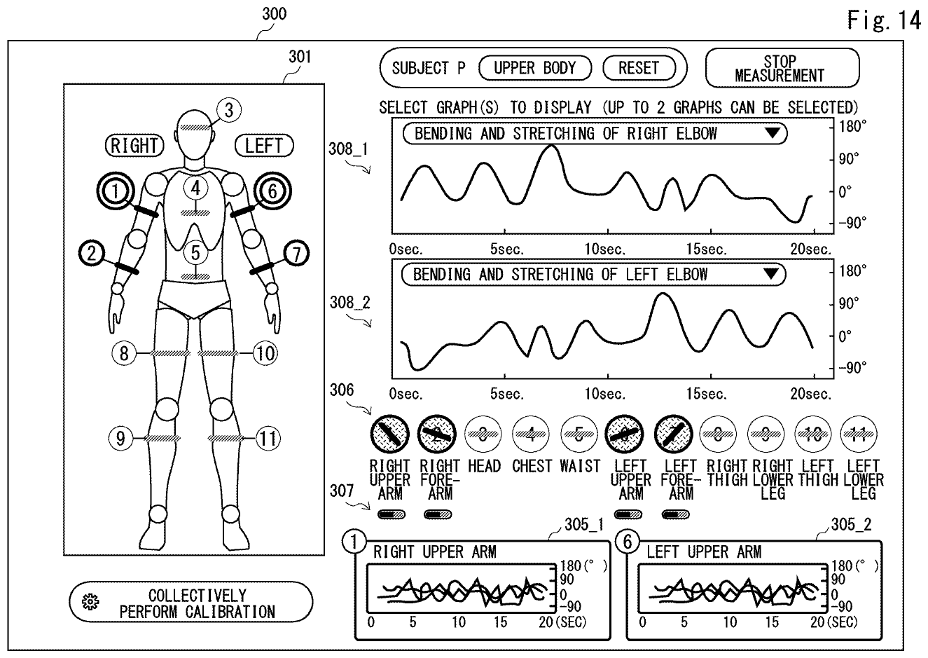

FIG. 13 is a diagram showing an example of a screen displayed on the monitor 300 after a calibration has been completed and before measurement of the motion to be monitored is started. FIG. 14 is a diagram showing an example of a screen displayed on the monitor 300 during the measurement of the motion to be monitored.

As shown in FIGS. 13 and 14, the monitor 300 displays at least the human body schematic diagram 301 of the subject, graphs 305_1 and 305_2 of respective results of detection (sensing values in the respective three axial directions) by two sensors selected by a user, a startup status 306 and a remaining battery power 307 of each sensor, and graphs 308_1 and 308_2 of results of calculations indicating motion states of two motions to be monitored selected by a user.

In the examples shown in FIGS. 13 and 14, the result of detection performed by the sensor 111_1 attached to the body part “1” (the body part 20_1) of the right upper arm is displayed as the graph 305_1, and the result of detection performed by the sensor 111_6 attached to the body part “6” (the body part 20_6) of the left upper arm is displayed as the graph 305_2. Further, in the examples shown in FIGS. 13 and 14, the result of a calculation indicating the motion state of the “bending and stretching of the right elbow” which is one of the motions to be monitored is displayed as the graph 308_1, and the result of a calculation indicating the motion state of the “bending and stretching of the left elbow” which is one of the motions to be monitored is displayed as the graph 308_2. The contents which these graphs show can be freely selected by a user.

Note that the monitor 300 may display all the graphs showing the respective results of detection performed by the four sensors 111_1, 111_2, 111_6, and 111_7. Further, the monitor 300 may display all the graphs showing the results of the calculations indicating the motion states of the four motions to be monitored.

Further, the graphs 308_1 and 308_2 showing the motion states of the motions to be monitored may be displayed so that they are each displayed in a size larger than that of information (e.g., the startup status 306 of each sensor, the remaining battery power 307 of each sensor, and the graphs 305_1 and 305_2 showing the results of detection performed by the sensors) about the sensor. Thus, it is possible to more easily visually recognize the motion state of the subject P.

Note that the result of the calculation indicating the motion state of the “bending and stretching of the right elbow” can be determined by, for example, a difference between the result of detection performed by the sensor 111_1 attached to the right upper arm and the result of detection performed by the sensor 111_2 attached to the right forearm. Therefore, the calculation processing unit 122 generates a result of the calculation indicating the motion state of the “bending and stretching of the right elbow” based on the result of detection performed by each of the sensors 111_1 and 111_2 selected by the selection unit 121. Then the output unit 123, which is the display apparatus, graphs and displays the result of the calculation generated by the calculation processing unit 122 on the monitor 300.

Further, the result of the calculation indicating the motion state of the “bending and stretching of the left elbow” can be determined by, for example, a difference between the result of detection performed by the sensor 111_6 attached to the left upper arm and the result of detection performed by the sensor 111_7 attached to the left forearm. Therefore, the calculation processing unit 122 generates a result of the calculation indicating the motion state of the “bending and stretching of the left elbow” based on the result of detection performed by each of the sensors 111_6 and 111_7 selected by the selection unit 121. Then the output unit 123, which is the display apparatus, graphs and displays the result of the calculation generated by the calculation processing unit 122 on the monitor 300.

Similarly, the result of the calculation indicating the motion state of the “internal and external rotation of the right shoulder” can be determined by, for example, a difference between the result of detection performed by the sensor 111_1 attached to the right upper arm and the result of detection performed by the sensor 111_2 attached to the right forearm. Therefore, the calculation processing unit 122 generates a result of the calculation indicating the motion state of the “internal and external rotation of the right shoulder” based on the result of detection performed by each of the sensors 111_1 and 111_2 selected by the selection unit 121. Then the output unit 123, which is the display apparatus, can graph and display the result of the calculation generated by the calculation processing unit 122 on the monitor 300.

Similarly, the result of the calculation indicating the motion state of the “internal and external rotation of the left shoulder” can be determined by, for example, a difference between the result of detection performed by the sensor 111_6 attached to the left upper arm and the result of detection performed by the sensor 111_7 attached to the left forearm. Therefore, the calculation processing unit 122 generates a result of the calculation indicating the motion state of the “internal and external rotation of the left shoulder” based on the result of detection performed by each of the sensors 111_6 and 111_7 selected by the selection unit 121. Then the output unit 123, which is the display apparatus, can graph and display the result of the calculation generated by the calculation processing unit 122 on the monitor 300.

As described above, the motion state monitoring apparatus 12 according to this embodiment and the training support system 1 which includes this motion state monitoring apparatus 12 output a result of a calculation indicating a motion state of the subject based on a result of detection performed by each of one or a plurality of sensors corresponding to the motions to be monitored among a plurality of sensors associated with a plurality of respective body parts. By this configuration, the motion state monitoring apparatus 12 according to this embodiment and the training support system 1 which includes this motion state monitoring apparatus 12 can output a more accurate result of a calculation indicating a motion state of the subject than when a result of detection performed by a set of sensors attached to one body part is used. As a result, a user can effectively monitor a complicated motion state of the subject. Further, the motion state monitoring apparatus 12 according to this embodiment and the training support system 1 which includes this motion state monitoring apparatus 12 can output a more accurate result of a calculation by using a result of detection performed by the sensor for which a calibration has been completed. As a result, a user can more accurately monitor the motion state of the subject.

Note that the order of the processes performed in the training support system 1 is not limited to the order of the processes shown in FIG. 6. For example, a calibration may be performed prior to pairing.

### <Modified Example of Training Support System 1>

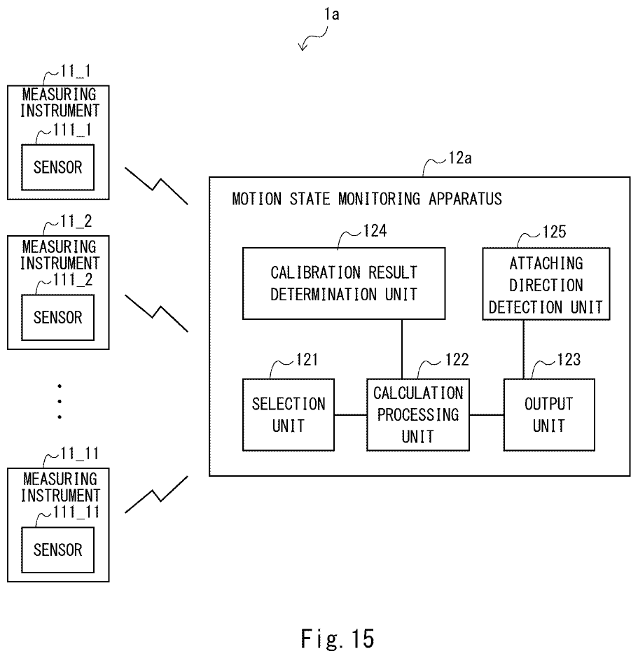

FIG. 15 is a block diagram showing a training support system 1a which is a modified example of the training support system 1. In the training support system 1a, unlike the training support system 1, each measuring instrument 11_1 to 11_11 is configured so that a direction with respect to the attachment pad 112_1 (hereinafter referred to as an attaching direction) in which the sensor is attached can be changed. Further, the training support system 1a includes a motion state monitoring apparatus 12a instead of the motion state monitoring apparatus 12. In addition to including the components included in the motion state monitoring apparatus 12, the motion state monitoring apparatus 12a further includes an attaching direction detection unit 125. Since the configurations of the motion state monitoring apparatus 12a other than the above ones are similar to those of the motion state monitoring apparatus 12, the descriptions thereof will be omitted.

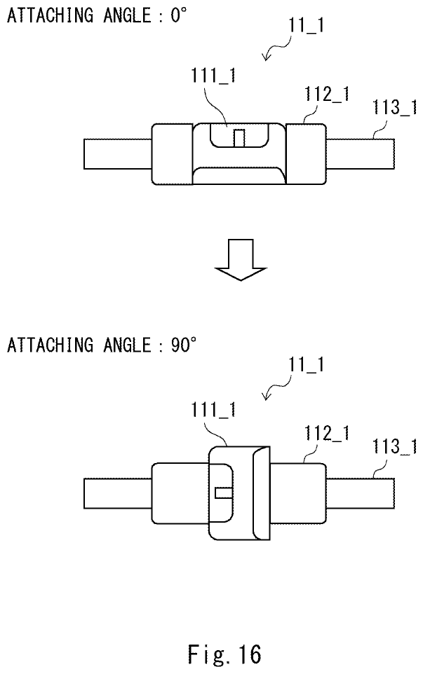

FIG. 16 is a diagram showing a configuration example of the measuring instrument 11_1 provided in the training support system 1a. Note that since the configuration of each of the measuring instruments 11_2 to 11_11 is similar to that of the measuring instrument 11_1, the descriptions thereof will be omitted.

As shown in FIG. 16, in the measuring instrument 11_1, the sensor 111_1 can be attached in any direction with respect to the attachment pad 112_1. If the direction of the sensor 111_1 when the sensor 111_1 is attached so that the longitudinal direction thereof is placed along the circumferential direction of the belt 113_1 is a reference attaching direction (an attaching angle is zero degrees), the sensor 111_1 can also be attached, for example, by rotating it 90 degrees with respect to the reference attaching direction. The measuring instrument 11_1 transmits, in addition to a result (a sensing value) of detection performed by the sensor 111_1, information about the attaching direction of the sensor 111_1 with respect to the reference attaching direction to the motion state monitoring apparatus 12a.

The attaching direction detection unit 125 is configured so that it can detect information about the attaching directions of the sensors 111_1 to 111_11 with respect to the respective reference attaching directions. The output unit 123 outputs information about the attaching direction of the sensor with respect to the reference attaching direction thereof detected by the attaching direction detection unit 125 together with the result of detection performed by the sensor, and outputs the result of detection performed by the sensor in which the attaching direction of the sensor has been taken into account. By doing so, a user can more accurately grasp the result of detection performed by the sensor.

As described above, the motion state monitoring apparatus according to the aforementioned embodiment and the training support system which includes this motion state monitoring apparatus output a result of a calculation indicating a motion state of the subject based on a result of detection performed by each of one or a plurality of sensors corresponding to the motions to be monitored among a plurality of sensors associated with a plurality of respective body parts. By this configuration, the motion state monitoring apparatus according to the aforementioned embodiment and the training support system which includes this motion state monitoring apparatus can output a more accurate result of a calculation indicating a motion state of the subject than when a result of detection performed by a set of sensors attached to one body part is used. As a result, a user can effectively monitor a complicated motion state of the subject. Further, the motion state monitoring apparatus according to the aforementioned embodiment and the training support system which includes this motion state monitoring apparatus can output a more accurate result of a calculation by using a result of detection performed by the sensor for which a calibration has been completed. As a result, a user can more accurately monitor the motion state of the subject.

Further, although the present disclosure has been described as a hardware configuration in the aforementioned embodiment, the present disclosure is not limited thereto. In the present disclosure, control processing of the motion state monitoring apparatus can be implemented by causing a Central Processing Unit (CPU) to execute a computer program.

Further, the above-described program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A motion state monitoring system comprising:
a selection unit configured to select one or a plurality of sensors from among a plurality of sensors associated with a plurality of respective body parts of a body of a subject based on one or a plurality of specified motions to be monitored;
a calibration result determination unit configured to determine whether or not a calibration of each of at least the one or plurality of sensors selected by the selection unit has been completed;
a calculation processing unit configured to generate a result of a calculation indicating a motion state of the subject based on a result of detection performed by each of the one or plurality of sensors selected by the selection unit when the calibration result determination unit determines that the calibration has been completed; and
an output unit configured to output the result of the calculation performed by the calculation processing unit.

2. The motion state monitoring system according to claim 1, wherein the calibration result determination unit is configured to determine that the calibration has been completed when an output value of each of at least the one or plurality of sensors selected by the selection unit falls within a predetermined range after a predetermined period of time has elapsed from when the calibration of each of at least the one or plurality of sensors is started.

3. The motion state monitoring system according to claim 2, wherein a time at which the calibration is started is a time at which an instruction that the calibration is to be started has been given in a state in which each of at least the one or plurality of sensors selected by the selection unit has been brought to a standstill.

4. The motion state monitoring system according to claim 2, wherein the time at which the calibration is started is a time at which power of each of at least the one or plurality of sensors selected by the selection unit is turned on in a state in which each of at least the one or plurality of sensors has been brought to a standstill.

5. The motion state monitoring system according to claim 1, wherein the output unit is further configured to output, when the calibration of one of at least the one or plurality of sensors selected by the selection unit is not completed, information prompting a user to bring the sensor for which the calibration is not completed to a standstill.

6. The motion state monitoring system according to claim 1, wherein the calibration result determination unit is further configured to determine whether or not all the calibrations of the plurality of sensors associated with the plurality of respective body parts of the body of the subject have been completed.

7. A training support system comprising:
a plurality of measuring instruments each comprising one of the plurality of sensors associated with a plurality of respective body parts of a body of a subject; and
the motion state monitoring system according to claim 1.

8. A method for controlling a motion state monitoring system, the method comprising:
selecting one or a plurality of sensors from among a plurality of sensors associated with a plurality of respective body parts of a body of a subject based on one or a plurality of specified motions to be monitored;
determining whether or not a calibration of each of at least the one or plurality of selected sensors has been completed;
generating a result of a calculation indicating a motion state of the subject based on a result of detection performed by each of the one or plurality of selected sensors when it is determined that the calibration has been completed; and
outputting the result of the calculation.

9. A non-transitory computer readable medium storing a control program for causing a computer to:
select one or a plurality of sensors from among a plurality of sensors associated with a plurality of respective body parts of a body of a subject based on one or a plurality of specified motions to be monitored;
determine whether or not a calibration of each of at least the one or plurality of selected sensors has been completed;
generate a result of a calculation indicating a motion state of the subject based on a result of detection performed by each of the one or plurality of selected sensors when it is determined that the calibration has been completed; and
output the result of the calculation.
